(19) United States
(12) Patent Application Publication
(10) Pub. No.: US 2022/0242895 A1
(43) Pub. Date: Aug. 4, 2022

(54) LIQUID PHASE SEPARATION OF 2G SUGARS BY ADSORPTION ON A FAU ZEOLITE HAVING A SI/AL ATOMIC RATIO GREATER THAN 1.5

(71) Applicant: IFP Energies nouvelles, Rueil-Malmaison Cedex (FR)

(72) Inventors: Catherine LAROCHE, Rueil-Malmaison Cedex (FR); Maria MANKO, Rueil-Malmaison Cedex (FR); Emmanuelle BRACCO, Rueil-Malmaison Cedex (FR)

(73) Assignee: IFP Energies nouvelles, Rueil-Malmaison Cedex (FR)

(21) Appl. No.: 17/621,987

(22) PCT Filed: Jun. 11, 2020
(86) PCT No.: PCT/EP2020/066154

§ 371 Date: Dec. 22, 2021

(30) Foreign Application Priority Data
Jun. 28, 2019 (FR) FR1907089

(51) Int. Cl. C07H 1/06; C07H 3/02; B01D 15/26

(52) U.S. Cl. CPC C07H 1/06; B01D 15/185; B01D 15/265; C07H 3/02

(57) ABSTRACT

The invention relates to a process for the liquid-phase separation of glucose from a mixture of C5 and C6 sugars comprising at least xylose and glucose, by adsorption of glucose on a zeolitic adsorbent based on FAU-type zeolite crystals having an Si/Al atomic ratio strictly greater than 1.5 comprising barium, wherein:
said mixture is brought into contact with said adsorbent, by liquid chromatography, to obtain a xylose-enriched liquid phase and a glucose-enriched adsorbed phase;
on the one hand, said xylose-enriched liquid phase is recovered and said phase adsorbed on said adsorbent is desorbed by means of a desorption solvent in order to recover the glucose on the other hand.

### TECHNICAL FIELD

The invention relates to the use of adsorbents based on agglomerated crystals of FAU-type zeolite with an Si/Al atomic ratio greater than 1.5, comprising barium for the liquid-phase separation of “second generation” (2G) C5 and C6 sugars, that is to say comprising respectively 5 or 6 carbon atoms, more particularly glucose and xylose.

### PRIOR ART

The production of “second generation” sugars from lignocellulosic biomass leads to a mixture of C5 and C6 sugars, essentially consisting of xylose (C5 sugar) and glucose (C6 sugar).

The stream of C5 and C6 sugars can be converted by fermentation into biobased molecules, of the alcohol (ethanol, n-propanol used as solvent in pharmacology) or diol type (Sheldon, 2018). For chemical conversion, a step of separating the sugars into C5 and C6 is necessary to enable them to be used in targeted reactions. Glucose (C6) and xylose (C5), being platform molecules, are converted into high value-added bioproducts. Glucose can be converted into sorbitol (used for personal care products), vitamin C, and then into isosorbide, a biodegradable monomer used for the production of polyethylene terephthalate for food use. Another example of the use of glucose is the isomerization thereof to fructose, which is the intermediate molecule for the exploitation of lignocellulosic biomass. Fructose can be dehydrated to hydroxymethylfurfural (5-HMF) which can be converted into biofuel, 2,5-dimethylfuran (DMF), or into acids such as furanodicarboxylic acid (FDCA), which is a precursor of the polymer polyethylene furanoate (PEF), and levulinic acid.

Today crystalline glucose is obtained by crystallization of high-glucose syrup (F. W. Schenck, 2012). Glucose syrup is obtained by enzymatic hydrolysis of starch, a polysaccharide present in first-generation (1G) biomass. However, the greatest use of starch is for the production of high-fructose-corn syrup (HFCS) used mainly in the agro-food industry. Starch-containing plants (wheat, corn, soft wheat) are edible and are in competition with the agro-food industry. Consequently, it is necessary to replace the glucose source with 2G biomass, and there is a need to obtain the purified glucose from the lignocellulosic material.

The prior art relating to the separation of xylose and glucose is quite poor. Two separation techniques are cited:
Membrane separation by nanofiltration: Sjöman et al. tested several nanofiltration membranes, including the NF270 membrane from Dow Liquid Separations, consisting of a semi-aromatic piperazine-based polyamide surface on a polysulfone support. On this type of membrane, the mode of separation is based on the difference in the size of the molecules, as xylose is smaller, it is less retained than glucose. The difference in retention varies depending on the total concentration and on the xylose: glucose ratio in the feedstock and also on the permeation rate.
Separation by adsorption: the technique is commonly used for the separation of sugars, and in particular for fructose/glucose separation, according to the simulated moving bed (SMB) process proposed in 1977 by Toray on an adsorbent based on X, Y and L zeolite (Odawara et al., 1979). U.S. Pat. No. 4,340,724 by Neuzil (1982) describes a selection of X and Y zeolites, with a list of exchange cations, for selective adsorption of fructose relative to glucose. The reference adsorbent for the fructose-glucose separation is a calcium-exchanged ion-exchange resin, of styrene-divinylbenzene (DVB) sulfonated copolymer type: this type of resin is offered by UOP for fructose-glucose separation in a simulated moving bed SMB (Landis et al; 1981). Such resins are now marketed and intended for the enrichment of High Fructose Corn Syrup (fructose-glucose separation) and for the purification of polyols in the food sector, for example the Cat exchanged Dowex 99 resin (Dow Water Solutions Brochure, No. 177-01566-0209). No information is available on glucose/xylose separation by selective adsorption on zeolite. In contrast, the Ca²⁺ exchanged Dowex 99 resin is disclosed in patent application US 2004/0173533 for xylose/glucose separation (Farrone, 2004), and shows a slightly greater retention of xylose relative to glucose on this resin. The xylose/glucose selectivity estimated from the pure substance isotherms measured by Lei et al. (J. Chem. Eng. Data, 2010) is however very low (between 1.188 and 1.399).

Despite this low selectivity, the evaluations by Vanneste et al. (Separation and Purification Technology, 2011), show that the separation by chromatographic process on Ca²⁺ exchanged styrene-DVB resin is economically comparable to separation by nanofiltration.

Zeolites, being crystalline materials, find their application in catalysis, adsorption and separation. Among more than two hundred zeolitic structures (http://www.iza-structure.org/databases) it is the FAU-type zeolite which is the most used in catalytic industrial processes such as the cracking of heavy petroleum cuts or in the separation of hydrocarbons, more particularly for the production of p-xylene from an aromatic feedstock containing C8 isomers. Faujasite, a FAU-type zeolite, exists in two forms: X form with an Si/Al atomic ratio of between 1 and 1.5 (R. M. Milton, 1959) and Y form with an Si/Al ratio of greater than 1.5 (D. W. Breck, 1964). Y-form faujasite-type zeolites, which find the main application in heterogeneous catalysis, can also be used for the separation of sugars (S. Kulprathipanja, 2017), without further clarification regarding their effectiveness for the separation of glucose and xylose.

Surprisingly, it appears that Y type agglomerated zeolitic adsorbents exchanged with barium have both a good adsorption capacity for sugars, which may optionally be improved by a step of zeolitization of the binder, and a favorable glucose/xylose selectivity, and may thus be successfully used in particular when they are produced from small crystals of FAU zeolite, in liquid-phase processes for the separation of glucose and xylose contained in “2^{nd} generation” sugar juices, for example of the simulated countercurrent type.

### SUMMARY OF THE INVENTION

The invention relates to a process for the liquid-phase separation of glucose from a mixture of C5 and C6 sugars comprising at least xylose and glucose, by adsorption of glucose on a zeolitic adsorbent based on FAU-type zeolite crystals having an Si/Al atomic ratio strictly greater than 1.5 comprising barium, wherein:
said mixture is brought into contact with said adsorbent, by liquid chromatography, to obtain a xylose-enriched liquid phase and a glucose-enriched adsorbed phase;
on the one hand, said xylose-enriched liquid phase is recovered and said phase adsorbed on said adsorbent is desorbed by means of a desorption solvent in order to recover the glucose on the other hand.

Said adsorbent may comprise zeolite crystals having a diameter of less than or equal to 2 μm, preferably less than or equal to 1 μm.

Preferably, the FAU zeolite has an Si/Al atomic ratio greater than or equal to 2.

Very preferably, the FAU zeolite has an Si/Al atomic ratio greater than or equal to 2.3.

Advantageously, the content of barium oxide BaO in said adsorbent is such that the Ba²⁺ exchange rate is greater than 50%, preferably greater than 60%, and more preferably greater than or equal to 65%.

Advantageously, said adsorbent has a total content of oxides of alkali metal or alkaline-earth metal ions other than barium, potassium and sodium, such that the exchange rate of all of said ions relative to all of the alkali metal or alkaline-earth metal ions, is less than 30%, preferably between 0% and 5%.

Advantageously, the separation by adsorption is carried out in a simulated moving bed: the xylose-enriched liquid phase is removed from contact with the adsorbent thus forming a raffinate stream, and the glucose-enriched phase adsorbed on said adsorbent is desorbed under the action of a desorption solvent, and removed from contact with the adsorbent then forming an extract stream.

Advantageously, the desorption solvent is water.

The separation by adsorption can be carried out in an industrial adsorption unit of simulated countercurrent type with the following operating conditions:
number of beds: 6 to 30,
at least 4 operating zones, each located between a feed point and a withdrawal point,
a temperature of from 20° C. to 100° C., preferably from 20° C. to 60° C., very preferably from 20° C. to 40° C.,
pressure of between atmospheric pressure and 0.5 MPa.

The adsorbent may be in the form of an agglomerate comprising a binder and the number-average diameter of the agglomerates is from 0.4 to 2 mm, preferably between 0.4 and 0.8 mm.

### DESCRIPTION OF THE EMBODIMENTS

Throughout the description, the expressions “between . . . and . . . ” and “from . . . to . . . ” used in the present description should be understood as including each of the limits mentioned, unless noted otherwise.

The present invention relates to the use of adsorbents based on FAU zeolite having an Si/Al atomic ratio strictly greater than 1.5, comprising barium, for the separation of glucose from a sugar juice containing glucose and xylose, by a simulated countercurrent process.

It has in fact been observed that, surprisingly, a glucose/xylose selectivity of greater than 1.5 could be obtained with adsorbents based on FAU zeolite having an Si/Al atomic ratio strictly greater than 1.5, comprising barium.

The present invention relates to a process for the liquid-phase separation of glucose, preferably of the simulated countercurrent type, from a sugar juice containing glucose and xylose, and using adsorbents based on FAU zeolite having an Si/Al atomic ratio strictly greater than 1.5, comprising barium.

Adsorbents comprising zeolite crystals having a diameter of less than or equal to 2 μm, preferably less than or equal to 1 μm, are preferred. This is because small zeolite crystals generally provide better mass transfer than crystals of the same zeolite with a larger particle size, in particular due to the improved mass transfer. In addition to good selectivity properties with respect to the species to be separated from the reaction mixture, the adsorbent then exhibits good mass transfer properties making it possible to contribute to an efficient separation of the species in the mixture.

The FAU zeolite used is a zeolite comprising barium, having an Si/Al atomic ratio such that Si/Al>1.5, preferably greater than 2, very preferably greater than 2.3, in order to improve the selectivity toward glucose.

The exchange rate of a given cation is defined as the ratio between the number of moles of oxide M_{2/n}O of the cation Mⁿ⁺ considered and the number of moles of all of the alkali metal and alkaline-earth metal oxides.

The contents of barium and of alkali metal or alkaline-earth metal ions other than barium and sodium, expressed in the form of oxides, are as follows:
the content of barium oxide BaO is advantageously such that the Ba²⁺ exchange rate is greater than 50%, preferably greater than 60%, and more preferably greater than or equal to 65%;
the total content of oxides of alkali metal or alkaline-earth metal ions other than barium and sodium, is advantageously such that the exchange rate of all of these ions relative to all of the alkali metal or alkaline-earth metal oxides, is less than 30%, preferably between 0% and 5%.

The adsorbent used in the process can be prepared according to the following steps:

a) mixing the crystals of desired particle size of FAU zeolite having an Si/Al atomic ratio such that Si/Al>1.5, preferably greater than 2, very preferably greater than 2.3, obtained by any method known to those skilled in the art, described in the literature (D. M. Ginter et al., 1992), in the presence of water with at least one binder based on a clay (kaolin type) or a mixture of clays, and optionally a source of silica;

b) shaping the mixture obtained in a) to produce agglomerates, then drying, optionally followed by a screening and/or cycloning step;

c) calcining the agglomerates obtained in b) at a temperature preferentially ranging from 500° C. to 600° C.;

d) optionally zeolitizing the binder by bringing the calcined agglomerates resulting from step c) into contact with an alkali metal basic aqueous solution, followed by washing;

e) ion exchange of the zeolitic agglomerates obtained in c) or in d) with barium ions, followed by washing and drying of the product thus treated.

The shaping step b) makes it possible to obtain zeolitic agglomerates having sufficient mechanical strength for the use thereof in a process for separating a liquid mixture in a simulated moving bed. However, the presence of binder reduces the proportion of active material in the adsorption sense, said active material being the FAU zeolite having an Si/Al atomic ratio such that Si/Al>1.5.

The optional step d) of zeolitizing the binder thus makes it possible to convert all or part of the binder into active material in the adsorption sense (FAU zeolite having an Si/Al atomic ratio such that Si/Al>1.5) in order to obtain binder-free agglomerates, i.e. no longer comprising a non-zeolitic phase (in an amount typically of less than 2%), or agglomerates with a low binder content, i.e. comprising little (an amount typically of between 2% and 5%) of non-zeolitic phase (generally non-zeolitized residual binder or any other amorphous phase after zeolitization) in the final agglomerate, while maintaining the mechanical strength.

The agglomerates resulting from step c/ or d/, whether they are in the form of beads or extrudates, generally have a number-average diameter ranging from 0.4 to 2 mm, and in particular between 0.4 and 0.8 mm, more preferably between 0.5 and 0.7 mm.

The present invention more particularly relates to a process for the liquid-phase separation of glucose from a sugar juice containing glucose and xylose, taking place by liquid chromatography, advantageously in a simulated moving bed, i.e. simulated countercurrent or simulated cocurrent moving bed, and more particularly simulated countercurrent moving bed.

The desorption solvent is preferably water.

In a liquid-phase adsorption separation process, the adsorbent solid is brought into contact with the liquid feed stream (feedstock) composed of a sugar juice containing xylose and glucose. By using a zeolitic adsorbent based on a zeolite of faujasite Y structure comprising barium, the glucose is then adsorbed in the micropores of the zeolite preferentially with respect to the xylose. The phase adsorbed in the micropores of the zeolite is then enriched in glucose relative to the initial mixture constituting the feed stream. Conversely, the liquid phase is enriched in xylose relative to the initial mixture constituting the feed stream.

When the process takes place in a simulated moving bed, the xylose-enriched liquid phase is then removed from contact with the adsorbent thus forming a raffinate stream, and the glucose-enriched adsorbed phase is desorbed under the action of a desorption stream (or desorption solvent), and removed from contact with the adsorbent then forming an extract stream.

In general, the operation of a simulated moving bed column can be described as follows:

A column comprises at least four zones, each of these zones being made up of a certain number of successive beds, and each zone being defined by its position between a feed point and a withdrawal point. Typically, a simulated moving bed unit for the separation of sugars is fed with at least one feedstock F to be fractionated (sugar juice) and one desorbent D, sometimes referred to as a desorption solvent or eluent (generally water), and at least one raffinate R containing the least selectively adsorbed feedstock products and desorbent and an extract E containing the most adsorbed feedstock product and desorbent are withdrawn from said unit.

Conventionally, 4 different chromatographic zones are defined in a column operating in simulated countercurrent mode.
Zone 1: zone for desorption of the most adsorbed feedstock product, between the injection of the desorbent D and the withdrawal of the extract E.
Zone 2: zone for desorption of the least selectively adsorbed feedstock products, between the withdrawal of the extract E and the injection of the feedstock to be fractionated F.
Zone 3: zone for adsorption of the most adsorbed feedstock product, between the injection of the feedstock and the withdrawal of the raffinate R.
Zone 4: zone located between the withdrawal of the raffinate R and the injection of the desorbent D.

The operating conditions of an industrial adsorption unit of simulated countercurrent type are advantageously the following:
number of beds: 6 to 30,
at least 4 operating zones, each located between a feed point and a withdrawal point,
temperature of from 20° C. to 100° C., preferably from 20° C. to 60° C., very preferably from 20° C. to 40° C.,
pressure of between atmospheric pressure and 0.5 MPa.

One of the techniques of choice for characterizing the adsorption of molecules in the liquid phase on a porous solid is to carry out a breakthrough. In his book “Principles of Adsorption and Adsorption Processes”, Ruthven defines the technique of breakthrough curves as the study of the injection of a range of adsorbable constituents.

### BIBLIOGRAPHY

R. A. Sheldon, The Road to Biorenewables: Carbohydrates to Commodity Chemicals, ACS Sustainable Chem. Eng., vol. 6, p. 4464-4480, 2018
F. W. Schenck, Glucose and Glucose-containing Syrups, Ullmann's encyclopedia of industrial chemistry. Wiley-VCH, Verlag, 2006
E. Sjoman, M. Manttari, M. Nystrom, H. Koivviko, H. Heikkila, Separation of xylose grom glucose by nanofiltration from concentrated monosaccharide solutions, Journal of Membrane Science, vol. 292, (1-2), p. 106-115, 2007
Odawara et al., U.S. Pat. No. 4,157,267, 1979
Neuzil, U.S. Pat. No. 4,340,724, 1982
Landis, Broughton, Fickel, U.S. Pat. No. 4,293,346, 1981
Brochure Dow Water Solutions, Dowex™ Monosphere™, Chromatographic Separation of Fructose and Glucose with Dowex Monosphere Ion Exchange Resin, Technical Manual, No. 177-01566-0209
Farrone, US 2004173533, 2004
H. Lei, Z. Bao, H. Xing, Y. Yang, Q. Ren, M. Zhao, H. Huang, Adsorption Behavior of Glucose, Xylose and Arabinose on Five Different Cation Exchange Resins, J. Chem. Eng. Data, vol. 55, (2), p. 735-738, 2010
J. Vanneste, S. de Ron, S. Vandecruys, S. A. Soare, S. Darvishmanesh, B. van der Bruggen, Techno-economic evaluation of membrane cascades relative to stimulated moving bed chromatography for the purification of mono-and oligosaccharides, Separation and Purification Technology 80, 600-609, 2011
http://www.iza-structure.org/databases
R. M. Milton, U.S. Pat. No. 2,882,244, 1959
D. W. Breck, U.S. Pat. No. 3,130,007, 1964
S. Kulprathipanja, Zeolites in Industrial Separation and Catalysis; Wiley-VCH, Verlag, 2010
D. M. Ginter, A. T. Bell, C. J. Radke, in Synthesis of Microporous Materials, Vol. 1, Molecular Sieves, M. L Occelli, H. E Robson (eds.), Van Nostrand Reinhold, New York, 1992, p. 6.
D. M. Ruthven, Principles of adsorption & adsorption processes, John Wiley & Sons, 1984

### Example 1

Several types of adsorbents are prepared: zeolitic adsorbents based on Y zeolite (A to D), ion-exchange resin (E).

The characteristics of the adsorbents are as follows:
A: Adsorbent based on Y zeolite having an Si/Al atomic ratio equal to 2.74 exchanged with barium such that the barium exchange rate is 65%, the other cations present being sodium, used in the form of a 0.6 mm diameter bead.
B: Adsorbent based on Y zeolite in sodium form having an Si/Al atomic ratio equal to 2.74, used in the form of a 0.6 mm diameter bead.
C: Adsorbent based on Y zeolite having an Si/Al atomic ratio equal to 2.74 exchanged with calcium such that the calcium exchange rate is 90%, the other cations present being sodium, used in the form of a 0.6 mm diameter bead.
D: Adsorbent based on Y zeolite having an Si/Al atomic ratio equal to 2.74 exchanged both with barium and potassium such that the exchange rate for each of the cations is 50%, used in the form of a 0.6 mm diameter bead.
E: Dowex® 99 resin initially with calcium, exchanged with barium

A breakthrough test (frontal chromatography) is performed with the adsorbents A to E to evaluate their efficiency.

The procedure for obtaining the breakthrough curves is as follows:
Filling a column of about 20 cm³ with the adsorbent and insertion in the test bench.
Filling with the solvent (water) at ambient temperature.
Gradual increase to the adsorption temperature (30° C.) under a stream of solvent with a flow rate of 0.5 cm³/min.
Solvent/feedstock changeover to inject the feedstock with a flow rate of 0.5 cm³/min.
Online Raman analysis of the effluent and optional collection for offline analysis by other techniques for analyzing sugars (HPLC, etc.).

The injection of the feedstock is maintained for a sufficient time for the composition of the effluent to correspond to the composition of the feedstock.

The pressure is sufficient for the feedstock to remain in liquid phase at the adsorption temperature (30° C.), i.e. 0.12 MPa.

The composition of the feedstock used for the tests is as follows:
glucose: 0.145 g/g
xylose: 0.145 g/g

The selectivity of the glucose (G) relative to the xylose (X) is calculated from the adsorbed mass quantities q_{G} and q_{X} of the two compounds (the latter being determined by material balance from the analysis of the breakthrough effluent) and of the composition of the feedstock (feedstock in which the mass fraction of the compounds is y_{G} and y_{X}):

$${α}_{G / X} = \frac{{q}_{G}}{{q}_{X}} \frac{{y}_{K}}{{y}_{G}} .$$

The breakthrough results are given in Table 1 below

TABLE 1

| | | Qads | | |
|---|---|---|---|---|
| | | Glucose | Qads Xylose | Glucose/Xylose |
| | | (g/g) | (g/g) | selectivity |
| According to the | A | 0.068 | 0.031 | 2.00 |
| invention | | | | |
| Comparative | B | 0.031 | 0.022 | 1.43 |
| Comparative | C | 0.023 | 0.015 | 1.25 |
| Comparative | D | 0.033 | 0.023 | 1.43 |
| Comparative | E | 0.038 | 0.040 | 1.00 |

Example 1 shows that the zeolitic adsorbent in accordance with the invention has improved properties of selectivity of glucose relative to xylose compared to the adsorbents or resins known from the prior art in the separation of sugars.

### Example 2

A: Adsorbent based on Y zeolite having an Si/Al atomic ratio equal to 2.74 exchanged with barium such that the barium exchange rate is 65%, the other cations present being sodium, used in the form of a 0.6 mm diameter bead.

The breakthrough tests (frontal chromatography) are performed with the adsorbent A at several temperatures to evaluate their efficiency.

The procedure for obtaining the breakthrough curves is as follows:
Filling a column of about 20 cm³ with the adsorbent and insertion in the test bench.
Filling with the solvent (water) at ambient temperature.
Gradual increase to the adsorption temperature (30° C., 40° C., 50° C., 60° C.) under a stream of solvent with a flow rate of 0.5 cm³/min.
Solvent/feedstock changeover to inject the feedstock with a flow rate of 0.5 cm³/min.
Online Raman analysis of the effluent and optional collection for offline analysis by other techniques for analyzing sugars (HPLC, etc.).

The injection of the feedstock is maintained for a sufficient time for the composition of the effluent to correspond to the composition of the feedstock.

The pressure is sufficient for the feedstock to remain in liquid phase at the adsorption temperature (30° C., 40° C., 50° C., 60° C.), i.e. 0.12 MPa.

The composition of the feedstock used for the tests is as follows:
glucose: 0.168 g/g
xylose: 0.120 g/g

The breakthrough results are given in Table 2 below

TABLE 2

| Adsorbent A: | | | |
|---|---|---|---|
| According to the | Qads | | |
| invention | Glucose | Qads Xylose | Glucose/Xylose |
| Temperature | (g/g) | (g/g) | selectivity |
| 30° C. | 0.077 | 0.021 | 2.57 |
| 40° C. | 0.072 | 0.019 | 2.70 |
| 50° C. | 0.066 | 0.018 | 2.71 |
| 60° C. | 0.062 | 0.017 | 2.69 |

The example shows that a zeolitic adsorbent in accordance with the invention has properties of selectivity of glucose relative to xylose which are favorable to the separation of glucose over the entire temperature range tested.

1. A process for the liquid-phase separation of glucose from a mixture of C5 and C6 sugars comprising at least xylose and glucose, by adsorption of glucose on a zeolitic adsorbent based on FAU-type zeolite crystals having an Si/Al atomic ratio strictly greater than 1.5 comprising barium, wherein:
said mixture is brought into contact with said adsorbent, by liquid chromatography, to obtain a xylose-enriched liquid phase and a glucose-enriched adsorbed phase;
on the one hand, said xylose-enriched liquid phase is recovered and said phase adsorbed on said adsorbent is desorbed by means of a desorption solvent in order to recover the glucose on the other hand.

2. The process as claimed in claim 1, wherein said adsorbent comprises zeolite crystals having a diameter of less than or equal to 2 μm, preferably less than or equal to 1 μm.

3. The process as claimed in claim 1, wherein the FAU zeolite has an Si/Al atomic ratio greater than or equal to 2.

4. The process as claimed in claim 3, wherein the FAU zeolite has an Si/Al atomic ratio greater than or equal to 2.3.

5. The process as claimed in claim 1, wherein the content of barium oxide BaO in said adsorbent is such that the Ba²⁺ exchange rate is greater than 50%, preferably greater than 60%, and more preferably greater than or equal to 65%.

6. The process as claimed in claim 1, wherein said adsorbent has a total content of oxides of alkali metal or alkaline-earth metal ions other than barium, potassium and sodium, such that the exchange rate of all of said ions relative to all of the alkali metal or alkaline-earth metal ions, is less than 30%, preferably between 0% and 5%.

7. The process as claimed in claim 1, wherein the separation by adsorption is carried out in a simulated moving bed: the xylose-enriched liquid phase is removed from contact with the adsorbent thus forming a raffinate stream, and the glucose-enriched phase adsorbed on said adsorbent is desorbed under the action of a desorption solvent, and removed from contact with the adsorbent then forming an extract stream.

8. The process as claimed in claim 1, wherein the desorption solvent is water.

9. The process as claimed in claim 7, wherein the separation by adsorption is carried out in an industrial adsorption unit of simulated countercurrent type with the following operating conditions:
number of beds: 6 to 30,
at least 4 operating zones, each located between a feed point and a withdrawal point,
a temperature of from 20° C. to 100° C., preferably from 20° C. to 60° C., very preferably from 20° C. to 40° C.,
pressure of between atmospheric pressure and 0.5 MPa.

10. The process as claimed in claim 1, wherein the adsorbent is in the form of an agglomerate comprising a binder and the number-average diameter of the agglomerates is from 0.4 to 2 mm, preferably between 0.4 and 0.8 mm.
